(19) United States
(12) Patent Application Publication
(10) Pub. No.: US 2022/0142593 A1
(43) Pub. Date: May 12, 2022

(54) COMPOSITE

(71) Applicant: Medibord Limited, Nottingham (GB)

(72) Inventors: Walter Berend Frank Boersma, Nottingham (GB); John Spencer Weightman, Nottingham (GB); Michael Ian Newton, Nottingham (GB); Robert H. Morris, Nottingham (GB); Christophe L. Trabi, Nottingham (GB)

(73) Assignee: Medibord Limited, Nottingham (GB)

(21) Appl. No.: 17/433,489

(22) PCT Filed: Feb. 25, 2020
(86) PCT No.: PCT/GB2020/050449

§ 371 Date: Aug. 24, 2021

(30) Foreign Application Priority Data
Feb. 25, 2019 (GB) 1902531.1

(51) Int. Cl. A61B 6/04; B32B 5/02; B32B 5/12; B32B 7/12; B32B 27/36; B32B 27/30

(52) U.S. Cl. CPC A61B 6/0442; B32B 5/024; B32B 5/12; B32B 7/12; B32B 2535/00; B32B 27/302; B32B 2262/148; B32B 2262/062; B32B 27/36

(57) ABSTRACT

The present invention relates to a patient board suitable for supporting a patient during medical imaging, radiotherapy and/or surgery. The board comprises a composite comprising a natural or naturally-derived fibre and a thermoset matrix.

### CROSS-REFERENCE TO RELATED APPLICATIONS

This application is a U.S. National Phase filing of International Application No. PCT/GB2020/050449, filed Feb. 25, 2020, which relates and claims priority to Great Britain Application Number 1902531.1, filed Feb. 25, 2019, the entirety of each of which are hereby incorporated by reference in their entireties.

### BACKGROUND

The invention relates to a composite, particularly to a composite for a patient board for medical imaging, radiotherapy treatment and/or surgery.

Patient boards or tables provide a surface for a patient to lie or sit whilst they are undergoing medical imaging or radiotherapy treatment.

There are a various types of medical treatment tables presently available.

Carbon fibre reinforced composites are used on patient boards for standard 2D planar X-ray imaging, X-Ray computed tomography (CT/CAT) imaging and megavoltage imaging. Carbon fibre composites are typically difficult to design and manufacture, particularly with patient boards comprising complex shapes. Due to the difficulty in design, carbon fibre patient boards must be specifically and individually designed for each type of medical diagnosis or treatment equipment to be used resulting in high tooling costs as well as significant raw material costs.

Modifications to carbon fibre composites are difficult to make and require specialist equipment, and hence adapting the patient boards to a variety of machines is impractical. Furthermore, carbon fibre tables cannot readily be used with MRI equipment because the inherent electrical conductivity of the material generates potentially harmful currents in the presence of switched magnetic fields. The currents cause localised heating, as well as noise and artefacts on the MRI images. Carbon fibre composites have a relatively high radiation absorption coefficient (i.e. attenuates the radiation from the imaging device) compared with other materials, which reduces the contrast of the patient images in the area of the patient board. This type of patient board is also not recyclable.

Due to the inherent problems with carbon fibre composites, glass reinforced composites are typically used in applications using magnetic resonance imaging (MRI). However, glass reinforced composites typically have poorer structural properties (such as mechanical stiffness) than carbon fibre composites and also suffer from a relatively high radiation absorption coefficient. This type of patient board is also not recyclable.

Other options include using a honeycomb structure in the core of the patient board. This provides an increased strength, however, the honeycomb structure provides an inhomogeneous X-ray image (i.e. the honeycombs are visible in the X-ray image). This creates artefacts in the X-ray image and can interfere with the interpretation of the image of the patient, particularly when planar 2D imaging is performed.

U.S. Pat. No. 9,131,871 discloses an example medical tabletop structure having a cellular honeycomb structure thermally fused in a sandwich configuration between two-fibre-reinforced polypropylene face sheets.

It is an objective of the present invention to mitigate one or more of the above problems with prior art patient boards. It is an additional or alternative objective of the present invention to provide a patient board which is compatible with multiple diagnostic/treatment modalities.

### SUMMARY OF THE DISCLOSURE

According to an aspect of the invention, there is provided a patient board as defined by claim 1.

According to a second aspect, there is provided medical imaging, radiotherapy and/or surgery apparatus comprising the patient board of the first aspect.

According to a third aspect, there is provided a composite material for medical imaging, radiotherapy and/or surgery applications comprising a natural fibre/semi-synthetic fibre and a thermoset matrix.

According to a further aspect of the invention, there is provided a composite comprising fibres derived from natural material (e.g. wood pulp) and a rigid or semi rigid polymer network, such that the composite allows for significant quantities of ionising and electromagnetic radiations to pass unimpeded therethrough (i.e. is significantly radio-translucent).

The fibres may be provided in the form of a fabric. The fabric may comprise or consist of said fibres.

Preferably, the fibres are provided in a tightly woven fabric. The fabric utilises fine yarn to produce a high quality, repeatable, homogenous weave.

The fibres may be less than 100 μm in diameter.

Preferably, the wood pulp derived fibres are produced using non-toxic organic compounds (for example N-Methylmorpholine N-oxide). This may provide an environmental benefit over conventional processing/production of fibres.

Preferably, the polymer matrix comprises a material specifically selected for properties of recyclability or other environmental benefits.

A laminate structure may be provided, e.g. of which the composite material comprises one or more layer.

The composite material may be directly laminated with a core material to bring structural rigidity but may otherwise be bonded with adhesive or using welding. This may improve the load bearing capacity of the resulting structure, e.g. for use as a patient board.

Additional materials to bring structural rigidity and/or to allow load bearing may be incorporated within the material of the composite material and/or laminate structure, e.g. at the time of production.

The composite may provide a planar board or patient support aid for medical imaging, therapy or surgical applications.

The composite may comprises supporting, alignment and/or positioning elements or features for medical imaging, therapy or surgical applications.

If a tightly woven fabric is used in the composite matrix, it may first be printed using a textile printing system to provide positioning, branding or any other information as desired.

Any essential or preferable feature defined in relation to any one aspect of the invention may be applied to any other aspect of the invention wherever practicable. Accordingly, various combinations of the above features or claims are to be accommodated by way of the disclosure herein.

### BRIEF DESCRIPTION OF THE DRAWINGS

FIG. 1 shows a composite 1 according to an embodiment of the invention provided in a laminate structure.

FIG. 2 shows a stress vs. strain curve for a composite skin according to an example of the present invention and a prior art composite.

FIG. 3 shows a comparative optical image and MRI image for a composite according to an example of the present invention.

FIG. 4 shows an X-ray image of a composite according to an example of the present invention.

FIG. 5 shows a megavoltage image of the present composite and a prior art composite.

FIG. 6 shows show the beam attenuation of a composite according to an example of the present invention and prior art composites.

### DETAILED DESCRIPTION OF THE DISCLOSURE

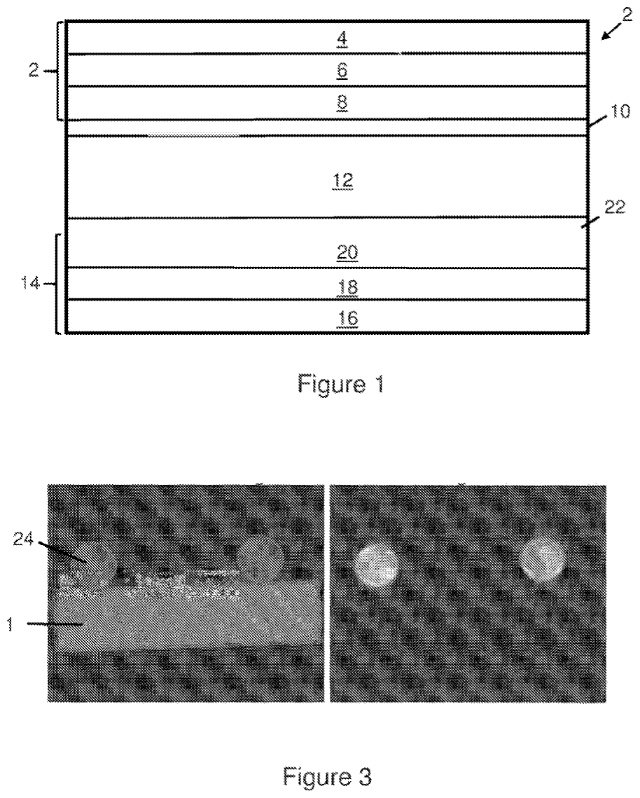

The composite 1 shown in FIG. 1 comprises a composite layer 2. The composite layer 2 comprises a composite of a natural fibre embedded within a thermoset polymer matrix. The natural fibre comprises a fibre sourced or derived from a natural source. For example, the natural fibre is sourced from an animal, plant or mineral fibre. In another example, the natural fibre is derived from a fibre, such as wood pulp, to provide a semi-synthetic material.

The fibre may comprise one or more of: a cellulosic fibre; a protein fibre; or a mineral fibre. The cellulosic fibre may comprise one or more of: cotton fibre; linen fibre; wood pulp fibre; or rayon fibre. The rayon fibre may comprise one or more of: viscose fibre; modal fibre; or lyocell fibre. The protein fibre may comprise one or more of a keratinous fibre, such as: wool fibre; or silk fibre.

In an embodiment, the fibre comprises a cellulosic fibre. The cellulosic fibre is derived from wood pulp. The cellulosic fibre is manufactured using non-toxic compounds. For example, N-methylmorpholine N-oxide is used as a solvent to dissolve the wood pulp.

The fibre may be blended with a further fibre to form a blend. The further fibre may comprise one or more of: natural fibre; semi-synthetic fibre; synthetic fibre. The further fibre may comprise one or more of: polyester or silk for example. The blend may comprise 70-99% of the natural fibre and 1-30% of the further fibre.

The natural fibres, and optionally further fibres, may be provided as a fabric, e.g. in a woven form. The natural fibres and/or further fibres comprise a fine yarn to produce a high quality, repeatable, homogenous weave. Alternatively, the natural fibres may be individual staple fibres/filaments embedded in the thermoset matrix.

The natural fibres are typically less than 100 μm in diameter.

The fibre is substantially electrically non-conductive. Preferably, the fibre has a conductivity of less than 1 Sm⁻¹

The thermoset comprises a thermosetting polymer (i.e. a resin). The thermosetting polymer may comprise one or more of: a polyester resin; polyurethane; an epoxy resin; vinylester resin; phenolic resin. The thermoset comprises material that is widely recyclable.

In some embodiments, the polymer matrix comprises a photosensitive polymer (e.g. a polymer that cures in response to radiation). For example, the matrix comprises a UV curing resin. The UV curing resin may comprise one or more of: acrylated epoxy; acrylated polyester; acrylated urethane; or acrylated silicone. The matrix may comprise a medium to low viscosity resin, thereby increasing the wetting of the fibres.

Only the UV curing resin which is successfully cured needs to move through the remaining process and the remainder can be recovered, thus potentially reducing waste material and minimising environmental impact.

In some embodiments, the matrix comprises or consists of a naturally derived material. The naturally derived resin may or may not be photosensitive (e.g. photo-curable). For example, the matrix may comprise one or more of: soy-oil based resin; lignin based resin; or glycerine based epoxy.

The composite layer 2 is substantially transparent/translucent to ionising electromagnetic radiation, particularly, to electromagnetic radiation used in medical imaging.

The composite layer 2 may comprise a plurality of layers 4, 6, 8. Between 2 and 8 layers may be provided. The plurality of layers 4, 6, 8 are arranged such that the fibres in each layer are orientated at a different angle with respect to an adjacent layer to increase isotropy of the composite layer 2. Preferably, the fibres in each layer are orthogonally orientated with respect to an adjacent layer. In other embodiments, each layer is orientated at 45 degrees relative to an adjacent layer.

In other examples, it may be possible to provide the desired fibre arrangement in a single layer, e.g. by producing a bespoke fabric pattern, e.g. a 2-dimensional or 3-dimensional pattern to ensure varying orientation of the fibres through the depth of the composite layer.

The composite layer 2 is bonded to a core layer 12. The core layer 12 provides additional structural rigidity to the composite to the layer 2. The core layer 12 may comprise a polymeric material. The polymeric material comprises one or more of: polystyrene, PVC, Polyethylene terephthalate, Polyurethane or Polyether ether ketone or similar foamed material.

The composite layer 2 may be bonded to the core layer 12 using a bonding layer 10. The bonding layer 10 may comprise one or more of: a polymeric material; an adhesive; or a compatibilising layer. The polymeric material may comprise one or more of: polyurethane; Methyl Methacrylate, Epoxy; or other resin or similar materials.

Alternatively, the composite layer 2 is welded to the core layer 12. Alternatively, the bonding layer could be fused with or moulded onto the composite layer at the time of production.

A second composite layer 14 may be provided on an opposing side of the core layer 12 to the first composite layer 2. The second composite layer 14 is substantially the same as the first composite layer 2. The second composite layer 14 may comprise a plurality of layers 18, 20, 22, which may be orthogonally orientated with respect to an adjacent layer. The second composite layer may comprise any of the features or properties described above with respect to the first composite layer.

A second bonding layer 22 may bond the second composite layer 14 to the core layer 12. The second bonding layer 22 may be substantially the same as the first bonding layer 22.

In an embodiment, either or both of the composite layer 2 and the second composite layer 14 comprise 90% lyocell fibres blended with 10% polyester or silk fibres, and the thermoset comprises polyester resin. The composite layer 2 and the second composite layer 14 are arranged in four or five orthogonally and/or 45 degree orientated layers. The core 12 comprises polystyrene and the composite layer 2 and the second composite layer 14 are bonded to the core 12 using polyurethane bonding layers 10, 22.

Other structural elements may be incorporated into/provided on the composite to increase the structural rigidity including one or more of: an internal tubular structure; an external supporting edge; a frame enclosing two or more edges of the board. The structural elements may comprise substantially the same materials as the composite layer 2.

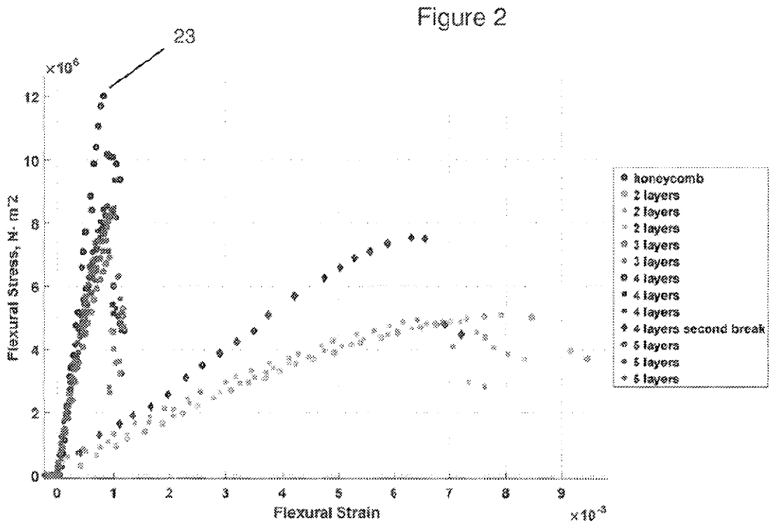

FIG. 2 shows the stress vs. strain curve for the composite 1 of the present invention and a prior art composite 23. A number of curves are shown according to the number of layers of composite 2 provided.

As shown in FIG. 2, a composite with 4 or 5 composite layers has comparable mechanical characteristics with the prior art composite 23 despite using intrinsically weaker natural fibres. Without being bound by theory, it is believed the natural fibres comprise imperfections which can key with the applied resin material. This may result in an increased mechanical bond between the fibres and material, e.g. so as to increases the fibre pull out strength of the composite. Whilst counterintuitive to use natural/semi-synthetic fibres of the type described herein, it has been found that they can provide surprising benefits for the specific applications disclosed herein. In some functional respects, the combination of a thermoset or resin material with woodpulp derived fibres has been found equivalent to a thermoplastic-based material. Natural fibre reinforcement such as hemp, jute, flax fibres or fibres derived therefrom could be used.

The composite 1 may comprise a board for medical imaging, therapy or surgery. The medical imaging techniques may include one of more of: magnetic resonance imaging (MRI);

Planar X-ray imaging; X-Ray computed tomography (CT/CAT) imaging; megavoltage imaging; or positron emission tomography (PET). The board is also be suitable for use in radiotherapy techniques or similar.

FIG. 3 shows an optical image (left) of the composite 1 adjacent a plurality of water-based location markers. The right image shows the same composite 1 and marker when viewed using MRI. As can be seen from the right image, the composite 1 has little or no interaction (i.e. a resonance) with the radiation/magnetism used in MRI and therefore, the composite is substantially transparent during MRI.

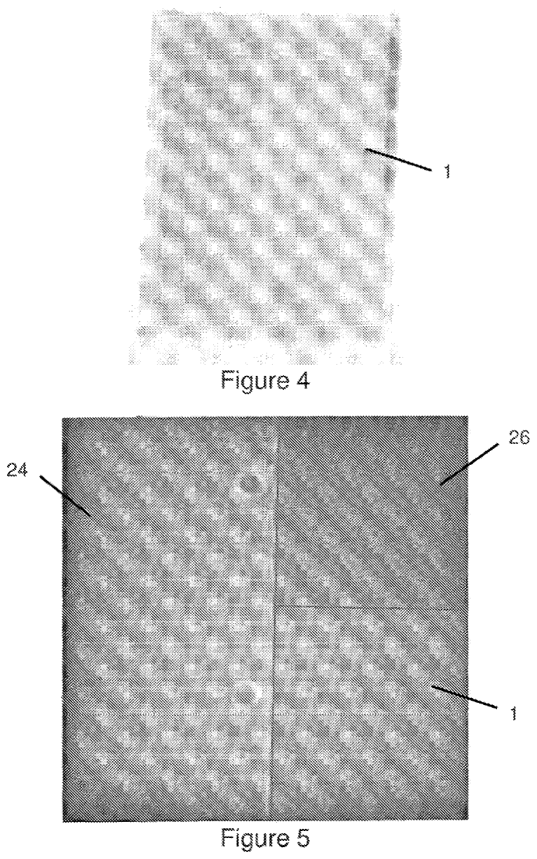

FIG. 4 shows an x-ray image of the composite 1 (looking down onto the top layer 2 of the composite 1). The composite 1 has a substantially uniform density and/or radiation absorptivity across the surface of the composite 1. The transparency of the board is substantially uniform and thus produces substantially homogenous images during X-ray imaging.

Similar results were obtained using a CT image.

FIG. 5 shows a megavoltage image of the composite 1 adjacent a first prior art board 24 and a second prior art board 26. It can be seen that the first prior art board produces a series of artefacts, including an internal honeycomb structure and a plurality of rings. However, composite 1 of the present invention has a substantially uniform density and/or radiation absorptivity about the surface of the composite 1, thus producing a substantially homogenous image.

The composite 1 produces substantially homogenous images during both X-ray, CT and megavoltage scans.

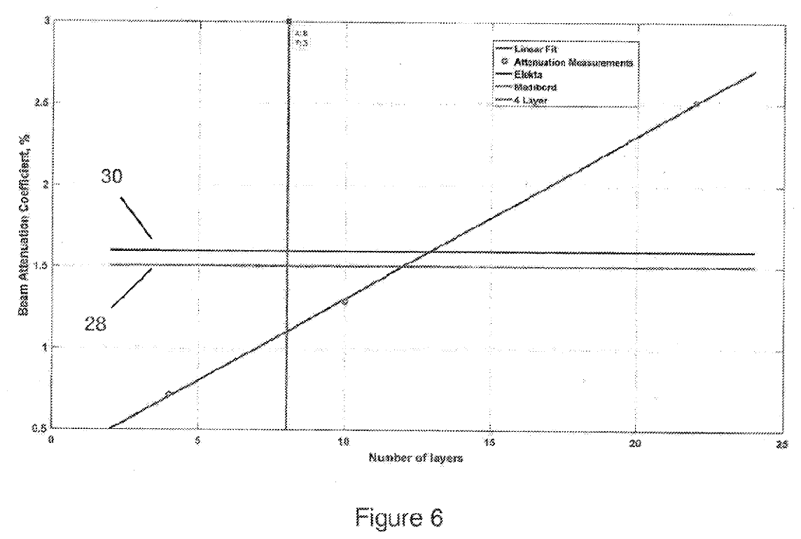

FIG. 6 shows the radiotherapy beam attenuation coefficient from a clinical linear accelerator (i.e. the amount of incident radiation absorbed) vs. the number of layers of material in the composite 1. A first prior art composite 28 and a second prior art composite 30 are shown for reference (the prior art composites have a fixed number of skin layers).

It can be seen from the graph, that when the composite 1 has fewer than 12 composite layers (e.g. 6 layers either side of the core), the composite has a reduced radiation attenuation coefficient (i.e. reduced the amount of radiation absorbed) compared with the prior art boards 28, 30.

The improved homogeneity and the reduced radiation attenuation coefficient of the composite 1 also provides greater imaging contrast and reduces visual artefacts, such that imaged features of the patient can be more easily discerned on planar or megavoltage X-Ray imaging.

The composite retains the structural strength of a prior art patient board; however, it is non-conductive and contains no carbon fibre and so is fully compatible with MRI apparatus. Therefore, the composite 1 is compatible with multiple types of imaging device, thus providing greater flexibility and mitigating the need to use separate boards for different apparatus. The board acts as a universal board, and a single board can be used on different apparatus throughout the complete diagnosis and treatment cycle of a patient, improving radiotherapy planning and treatment and thus patient outcomes.

In this way a board could remain with a patient through various different imaging and potentially therapy uses. This type of multimodal patient positioning board has not been hitherto possible and could be of significant benefit given the cost associated with the production of different boards for different uses.

The use of natural/cellulosic materials allows greater recyclability of the composite 1 and allows the composite 1 to be made from recycled materials. This reduces the carbon footprint and reduces the cost of disposal and therefore the overall environmental impact of the composite board.

In other examples, it is possible for the composite layer(s) to be provided as an insert or overlay for a base board material, rather than being bonded thereto. Suitable location features may be provided to ensure correct fitment. In this way, the composite layer may be removable from the base/substrate portion.

1. A patient board suitable for supporting a patient during medical imaging, radiotherapy and/or surgery, comprising:
a composite comprising a natural or naturally-derived fibre and a thermoset matrix.

2. A patient board according to claim 1, where the fibre comprises a cellulosic material.

3. A patient board according to claim 2, where the cellulosic material comprises lyocell.

4. A patient board according to claim 1, where the thermoset is a polyester resin.

5. A patient board according to claim 1, where the composite comprises a plurality of layers.

6. A patient board according to claim 5, where the number of layers is between 3 and 6.

7. A patient board according to claim 5, where the layers are one or more: orthogonally orientated; or oriented at 45 degrees with respective to an adjacent layer.

8. A patient board according to claim 1, where the composite is bonded to a core.

9. A patient board according to claim 8, where the core comprises polystyrene.

10. A patient board according to claim 8, where the composite is bonded to the core using a polyurethane layer.

11. A patient board according to claim 8, where a second composite is bonded to an opposing side of the core to the first composite.

12. A patient board according to claim 11, where the second composite comprises a plurality of layers.

13. A patient board according to claim 12, where the number of layers is between 3 and 6.

14. A patient board according to claim 11, where the second composite is bonded to the core using a polyurethane layer.

15. A patient board according to claim 1, where the fibre is woven.

16. A patient board according to claim 1, where fibre is blended with a further fibre comprising one or more of: a natural fibre; a semi-synthetic fibre; or a synthetic fibre, to form a blend.

17. A patient board according to claim 16, where the further fibre is polyester.

18. A patient board according to claims 16, where the blend comprises 70-99% of the fibre and 1-30% of the further fibre.

19. A patient board according to claim 1, where the thermoset comprises a photo-curable polymer.

20. A patient board according to claim 19, where the photo-curable polymer comprises a UV curable resin.

21. A patient board according to claim 1, where the thermoset comprises a naturally-derived polymer.

22. A patient board according to claim 1, where the thermoset comprises an epoxy resin.

23. A medical imaging/radiotherapy/surgery apparatus comprising the patient board of claim 1.

24. A composite structure comprising materials from claim 1, where the fabric has been directly printed with positioning, marketing or other information.

25. A composite for medical imaging/radiotherapy/surgery applications comprising a natural fibre/semi-synthetic fibre and a thermoset matrix.
